(19) United States
(12) Patent Application Publication
(10) Pub. No.: US 2026/0055064 A1
(43) Pub. Date: Feb. 26, 2026

(54) LOW-ODOR, HIGH-STABILITY ERGOTHIONEINE CRYSTAL

(71) Applicant: GENE III BIOTECHNOLOGY CO., LTD., Nanjing (CN)

(72) Inventors: Cong GUO, Nanjing (CN); Juan CAO, Nanjing (CN); Wei DING, Nanjing (CN); Yongxian TANG, Nanjing (CN)

(73) Assignee: GENE III BIOTECHNOLOGY CO., LTD., Nanjing (CN)

(21) Appl. No.: 19/298,619

(22) Filed: Aug. 13, 2025

(30) Foreign Application Priority Data
Aug. 23, 2024 (CN) 202411166971.3

(51) Int. Cl. C07D 233/84

(52) U.S. Cl. CPC C07D 233/84

(57) ABSTRACT

Provided is a low-odor, high-stability ergothioneine crystal, which belongs to the field of compound crystals The powder X-ray diffraction pattern of the ergothioneine crystal, measured using Cu-Kα radiation and expressed in terms of 2θ, has characteristic peaks at least at 9.50±0.2°, 15.52±0.2°, 19.06±0.2°, 24.88±0.2°, 25.48±0.2°, 28.74±0.2°, 29.22±0.2°, and 35.10±0.2°. The new crystal form of ergothioneine has excellent photostability, good resistance to hygroscopicity, and a high decomposition temperature tolerance.

### TECHNICAL FIELD

The present disclosure belongs to the field of compound crystals and, in particular, to a low-odor, high-stability ergothioneine crystal.

### BACKGROUND

Ergothioneine (EGT, also referred to as 2-mercaptohistidine betaine) is a white crystal as its pure product, and its molecular formula is C₉H₁₅N₃O₂S, with a molecular weight of 229.09.

Ergothioneine possesses an exceptional antioxidant capacity and can protect cells within the human body, functioning as an essential bioactive compound in organisms. It has multiple physiological functions, including scavenging free radicals, detoxification, maintaining DNA biosynthesis, supporting normal cell growth, and regulating cellular immune responses.

Ergothioneine exists in two isomeric forms: thiol isomers and thione isomers, as shown below:

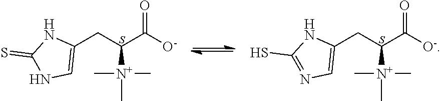

Under light conditions, ergothioneine may undergo degradation and develop an odor resembling trimethylamine. In addition, resistance to hygroscopicity of ergothioneine requires improvement.

Chinese Patent Application No. CN118221592A discloses an ergothioneine crystal and a preparation method thereof. The preparation method includes the following steps: crude ergothioneine with a purity of 98.0% to 99.9% is dissolved in warm water at 30° C. to 40° C. with stirring, cooled to 5° C. to 10° C., and allowed to stand to obtain an ergothioneine crystal. However, this preparation method fails to address the hygroscopic resistance and photostability issues of the product.

Currently, the existing art does not disclose new ergothioneine crystals having improved resistance to hygroscopicity and photostability.

Therefore, it is particularly important to develop a crystal form that is more resistant to light, more stable, and less susceptible to moisture absorption.

### SUMMARY

The present disclosure provides a low-odor, high-stability ergothioneine crystal.

A low-odor, high-stability ergothioneine crystal is provided. The powder X-ray diffraction (XRD) pattern of the ergothioneine crystal, measured using Cu-Kα radiation and expressed in terms of 2θ, has characteristic peaks at least at 9.50±0.2°, 15.52±0.2°, 19.06±0.2°, 24.88±0.2°, 25.48±0.2°, 28.74±0.2°, 29.22±0.2°, and 35.10±0.2°.

Preferably, the powder X-ray diffraction pattern of the ergothioneine crystal, measured using Cu-Kα radiation and expressed in terms of 2θ, also has characteristic peaks at 16.10±0.2°, 17.32±0.2°, 20.50±0.2°, 23.98±0.2°, 26.40±0.2°, and 38.62±0.2°.

Further preferably, the powder X-ray diffraction pattern of the ergothioneine crystal, measured using Cu-Kα radiation and expressed in terms of 2θ, also has characteristic peaks at 14.46±0.2°, 21.26±0.2°, 22.28±0.2°, 25.92±0.2°, 29.86±0.2°, 30.26±0.2°, 30.72±0.2°, 32.26±0.2°, 32.76±0.2°, 34.66±0.2°, 35.68±0.2°, 36.56±0.2°, 36.98-0.2°, 37.96±0.2°, and 39.24±0.2°.

Preferably, the characteristic peaks in the powder X-ray diffraction pattern (Cu-Kα) of the ergothioneine crystal are specifically shown in FIG. 7 and Table 1.

TABLE 1
Major XRD peaks of the ergothioneine crystal

| | Full width at | Interplanar | | Relative |
|---|---|---|---|---|
| Location | half maximum | spacing | Intensity | intensity |
| [2θ(°)] | [2θ(°)] | [Å] | [cps] | [%] |
| 9.500 | 0.188 | 9.3020 | 13689 | 15 |
| 14.460 | 0.259 | 6.1205 | 207 | 1 |
| 15.520 | 0.259 | 5.7048 | 4994 | 6 |
| 16.100 | 0.165 | 5.5005 | 1082 | 2 |
| 17.320 | 0.212 | 5.1157 | 2507 | 3 |
| 19.060 | 0.188 | 4.6525 | 92172 | 100 |
| 20.500 | 0.165 | 4.3288 | 1591 | 2 |
| 21.260 | 0.188 | 4.1757 | 376 | 1 |
| 22.280 | 0.188 | 3.9868 | 657 | 1 |
| 23.980 | 0.212 | 3.7079 | 1482 | 2 |
| 24.880 | 0.212 | 3.5758 | 6589 | 8 |
| 25.480 | 0.165 | 3.4929 | 4281 | 5 |
| 25.920 | 0.212 | 3.4346 | 258 | 1 |
| 26.400 | 0.235 | 3.3732 | 2249 | 3 |
| 28.740 | 0.212 | 3.1037 | 5391 | 6 |
| 29.220 | 0.235 | 3.0538 | 1730 | 2 |
| 29.860 | 0.165 | 2.9898 | 215 | 1 |
| 30.260 | 0.259 | 2.9512 | 224 | 1 |
| 30.720 | 0.212 | 2.9080 | 497 | 1 |
| 32.260 | 0.282 | 2.7726 | 597 | 1 |
| 32.760 | 0.212 | 2.7314 | 545 | 1 |
| 34.660 | 0.212 | 2.5859 | 187 | 1 |
| 35.100 | 0.188 | 2.5545 | 3787 | 5 |
| 35.680 | 0.165 | 2.5143 | 151 | 1 |
| 36.560 | 0.235 | 2.4558 | 477 | 1 |
| 36.980 | 0.235 | 2.4288 | 253 | 1 |
| 37.960 | 0.165 | 2.3684 | 263 | 1 |
| 38.620 | 0.235 | 2.3294 | 2695 | 3 |
| 39.240 | 0.353 | 2.2940 | 176 | 1 |

Preferably, a maximum mass loss temperature of the ergothioneine crystal is 302° C. to 305° C.

Preferably, a melting point of the ergothioneine crystal is 275° C. to 277° C.

Preferably, a method for preparing the ergothioneine crystal includes the following steps:
(1) mixing crude ergothioneine and water to prepare a crude solution of 70 g/L to 150 g/L, and adding ethanol or methanol to the crude solution for crystallization to obtain a crystal 1;
(2) mixing the crystal 1 with an aqueous solvent to prepare a solution A of 250 g/L to 500 g/L;
(3) adding ethanol or methanol to the solution A until crystal precipitation occurs, maintaining crystal growth, adding ethanol or methanol continuously, cultivating crystals, cooling, filtering and drying to obtain the ergothioneine crystal.

Further preferably, in step (1), an added amount of ethanol or methanol is 3 to 7 times the volume of the crude solution, crystallization is performed at a temperature of 20° C. to 50° C., crystallization is carried out for 0.5 hours to 1.5 hours, and crystallization is carried out with stirring at a rotational speed of 300 rpm to 800 rpm.

Further preferably, the aqueous solvent in step (2) includes water or the crude solution in step (1), and a total amount of ethanol or methanol added in step (3) is 3 to 7 times the volume of the crystal 1 solution.

Further preferably, in step (3), the temperature is controlled at 40° C. to 80° C. after ethanol or methanol is added, the crystal growth is maintained for 0.5 hours to 2.5 hours, ethanol or methanol is added with stirring at a rotational speed of 200 rpm to 500 rpm before crystal precipitation occurs, the crystal cultivation is performed for 0.5 hours to 3 hours, cooling is performed via gradient cooling at a rate of 10° C. per hour until reaching 25-50° C., and the crystal is dried at a temperature of 50° C. to 80° C.

The present disclosure further relates to use of the ergothioneine crystal in the preparation of food, a health product, a cosmetic or a drug.

Compared with the existing art, the present disclosure has the following beneficial effects.

(1) In the present disclosure, by optimizing the concentrations of the solutions in the secondary crystallization process, the new crystal form of ergothioneine finally prepared has excellent photostability, good resistance to hygroscopicity, and a high decomposition temperature tolerance.

(2) In the present disclosure, by optimizing the amounts of the alcohol solvents in the secondary crystallization process, the purity and the yield of the product are improved while ensuring better photostability and resistance to hygroscopicity.

### BRIEF DESCRIPTION OF DRAWINGS

FIG. 1 is a scanning electron microscope image of the new crystal form of ergothioneine of Example 1;

FIG. 2 is a scanning electron microscope image of the new crystal form of ergothioneine of Example 3;

FIG. 3 is a scanning electron microscope image of the new crystal form of ergothioneine of Example 4;

FIG. 4 is a scanning electron microscope image of the new crystal form of ergothioneine of Comparative Example 5;

FIG. 5 is a scanning electron microscope image of a commercially available ergothioneine product;

FIG. 6 is a powder X-ray diffraction pattern of the new crystal form of ergothioneine of Example 1;

FIG. 7 is a powder X-ray diffraction pattern of the new crystal form of ergothioneine of Example 3;

FIG. 8 is a powder X-ray diffraction pattern of the new crystal form of ergothioneine of Example 4;

FIG. 9 is a powder X-ray diffraction pattern of the new crystal form of ergothioneine of Example 5;

FIG. 10 is a powder X-ray diffraction pattern of the commercially available ergothioneine product;

FIG. 11 is a thermogravimetric analysis plot of the new crystal form of ergothioneine of Example 3; and

FIG. 12 is a thermogravimetric analysis plot of the commercially available ergothioneine product.

### DETAILED DESCRIPTION

The present disclosure is described below in detail in conjunction with specific examples. The examples set forth below are not intended to limit the present disclosure, but only to illustrate the present disclosure. The experimental methods used in the examples are conventional, unless otherwise specified; the experimental methods without specified conditions in the examples are generally conducted under conventional conditions; the materials, agents or the like used in the examples are generally commercially available, unless otherwise specified.

### Example 1

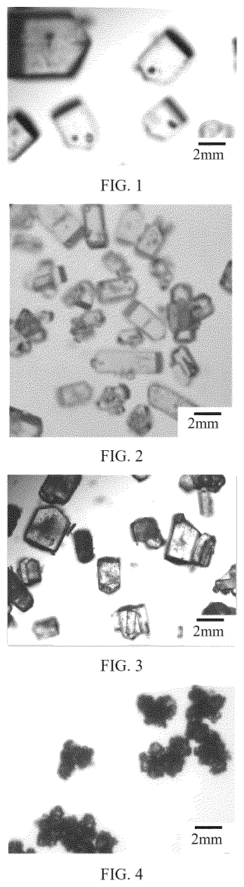

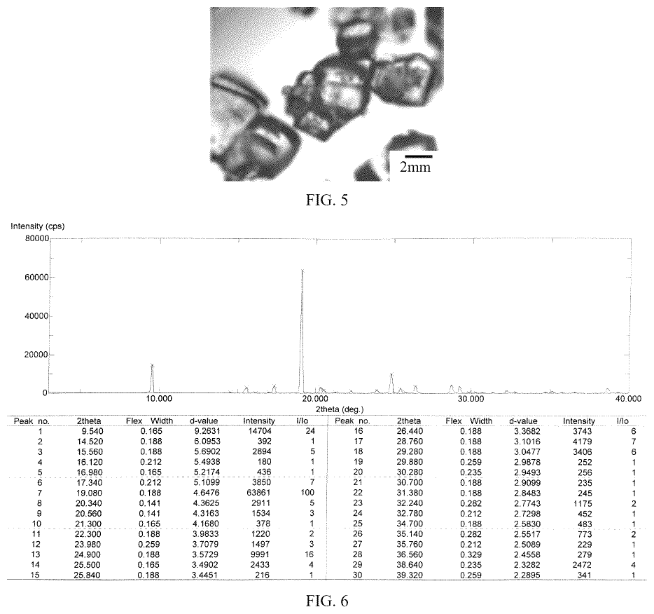

This example provides a low-odor, high-stability ergothioneine crystal. The scanning electron microscope image of the ergothioneine crystal is shown in FIG. 1, and the powder X-ray diffraction pattern, measured using Cu-Kα radiation, of the ergothioneine crystal is shown in FIG. 6.

The ergothioneine crystal was prepared in the following method.

(1) Crude ergothioneine was dissolved in water to prepare a crude ergothioneine solution of 70 g/L, methanol was added (the added amount of methanol was 5 times the volume of the crude ergothioneine solution), the temperature was controlled at 20° C., the crude ergothioneine solution was then stirred at a rotational speed of 300 rpm to carry out primary crystallization, and crystal growth was maintained for 0.5 hours to obtain a crystal 1.

(2) The crystal 1 was re-dissolved in water to obtain a solution A of 250 g/L, and the temperature was controlled at 40° C.

(3) Methanol was added to the solution A, the temperature was kept unchanged, the solution A was then stirred at a rotational speed of 200 rpm until crystal precipitation occurred, crystal growth was maintained for 0.5 hours, methanol was continuously added, the temperature was kept unchanged, the crystal cultivation was performed for 0.5 hours, the temperature was gradiently lowered at a rate of 10° C. per hour until the temperature was lowered to 25° C., and the crystal was collected by filtration and dried in an oven at 50° C. to obtain the ergothioneine crystal, where the total amount of methanol in two additions was 3.5 times the volume of the solution A.

The purity of the ergothioneine crystal was 99.97%, and the yield was 90%.

### Example 2

This example provides a low-odor, high-stability ergothioneine crystal. The ergothioneine crystal was prepared in the following method.

(1) Crude ergothioneine was dissolved in water to prepare a crude ergothioneine solution of 150 g/L, absolute ethanol was added (the added amount of absolute ethanol was 6.5 times the volume of the crude ergothioneine solution), the temperature was controlled at 50° C., the crude ergothioneine solution was then stirred at a rotational speed of 800 rpm to carry out primary crystallization, and crystal growth was maintained for 1.5 hours to obtain a crystal 1.

(2) The crystal 1 was re-dissolved in water to obtain a solution A of 500 g/L, and the temperature was controlled at 80° C.

(3) Absolute ethanol was added to the solution A, the temperature was kept unchanged, the solution A was then stirred at a rotational speed of 500 rpm until crystal precipitation occurred, crystal growth was maintained for 2.5 hours, absolute ethanol was continuously added, the temperature was kept unchanged, the crystal cultivation was performed for 3 hours, the temperature was gradiently lowered at a rate of 10° C. per hour until the temperature was lowered to 50° C., and the crystal was collected by filtration and dried in an oven at 80° C. to obtain the ergothioneine crystal, where the total amount of absolute ethanol in two additions was 6.5 times the volume of the solution A.

The purity of the ergothioneine crystal was 99.99%, and the yield was 92%.

### Example 3

This example provides a low-odor, high-stability ergothioneine crystal. The scanning electron microscope image of the ergothioneine crystal is shown in FIG. 2, and the powder X-ray diffraction pattern, measured using Cu-Kα radiation, of the ergothioneine crystal is shown in FIG. 7.

The ergothioneine crystal was prepared in the following method.

(1) Crude ergothioneine was dissolved in water to prepare a crude ergothioneine solution of 110 g/L, methanol was added (the added amount of methanol was 3.5 times the volume of the crude ergothioneine solution), the temperature was controlled at 35° C., the crude ergothioneine solution was then stirred at a rotational speed of 600 rpm to carry out primary crystallization, and crystal growth was maintained for 1 hour to obtain a crystal 1.

(2) The crystal 1 was re-dissolved in water to obtain a solution A of 400 g/L, and the temperature was controlled at 60° C.

(3) Methanol was added to the solution A, the temperature was kept unchanged, the solution A was then stirred at a rotational speed of 350 rpm until crystal precipitation occurred, crystal growth was maintained for 1.5 hours, methanol was continuously added, the temperature was kept unchanged, the crystal cultivation was performed for 2 hours, the temperature was gradiently lowered at a rate of 10° C. per hour until the temperature was lowered to 35° C., and the crystal was collected by filtration and dried in an oven at 70° C. to obtain the ergothioneine crystal, where the total amount of methanol in two additions was 5 times the volume of the solution A.

The purity of the ergothioneine crystal was 99.98%, and the yield was 91.5%.

### Example 4

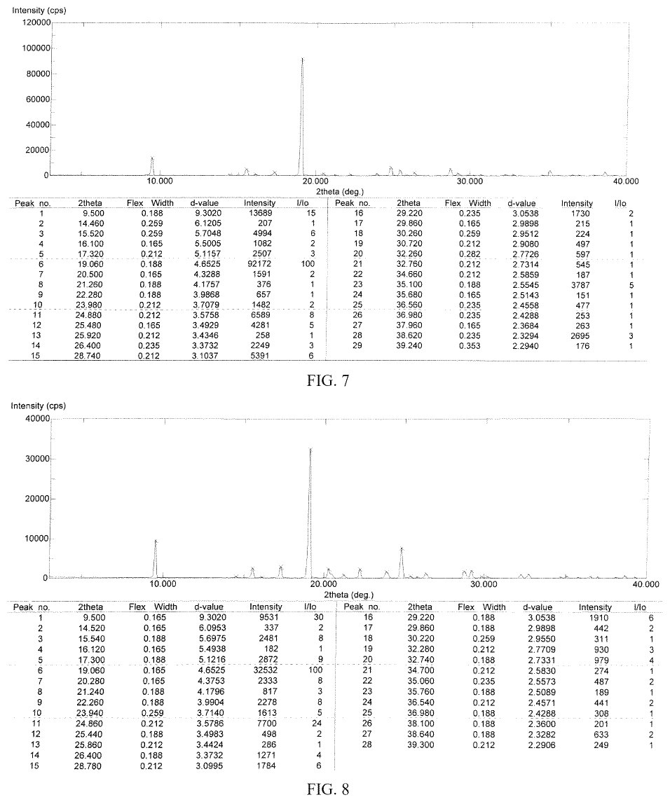

This example provides a low-odor, high-stability ergothioneine crystal. The scanning electron microscope image of the ergothioneine crystal is shown in FIG. 3, and the powder X-ray diffraction pattern, measured using Cu-Kα radiation, of the ergothioneine crystal is shown in FIG. 8.

The ergothioneine crystal was prepared in the following method.

(1) Crude ergothioneine was dissolved in water to prepare a crude ergothioneine solution of 120 g/L, methanol was added (the added amount of methanol was 3 times the volume of the crude ergothioneine solution), the temperature was controlled at 30° C., the crude ergothioneine solution was then stirred at a rotational speed of 500 rpm to carry out primary crystallization, and crystal growth was maintained for 1 hour to obtain a crystal 1.

(2) The crystal 1 was re-dissolved in the crude ergothioneine solution prepared in step (1) to obtain a solution A of 350 g/L, and the temperature was controlled at 70° C.

(3) Methanol was added to the solution A, the temperature was kept unchanged, the solution A was then stirred at a rotational speed of 300 rpm until crystal precipitation occurred, crystal growth was maintained for 2 hours, methanol was continuously added, the temperature was kept unchanged, the crystal cultivation was performed for 1.5 hours, the temperature was gradiently lowered at a rate of 10° C. per hour until the temperature was lowered to 40° C., and the crystal was collected by filtration and dried in an oven at 60° C. to obtain the ergothioneine crystal, where the total amount of methanol in two additions was 5.5 times the volume of the solution A.

The purity of the ergothioneine crystal was 99.96%, and the yield was 92.5%.

### Comparative Example 1

This comparative example differs from Example 3 only in that the concentration of the solution A in step (2) was 200 g/L, and the remaining conditions were the same.

The purity of the ergothioneine crystal was 99.99%, and the yield was 85%.

### Comparative Example 2

This comparative example differs from Example 3 only in that the concentration of the solution A in step (2) was 600 g/L, and the remaining conditions were the same.

The purity of the ergothioneine crystal was 95.96%, and the yield was 91.2%.

### Comparative Example 3

This comparative example differs from Example 3 only in that the total amount of methanol in two additions in step (3) was 3 times the volume of the solution A, and the remaining conditions were the same. The purity of the ergothioneine crystal was 98.24%, and the yield was 85%.

### Comparative Example 4

This comparative example differs from Example 3 only in that the total amount of methanol in two additions in step (3) was 7 times the volume of the solution A, and the remaining conditions were the same. The purity of the ergothioneine crystal was 94.15%, and the yield was 91.5%.

### Comparative Example 5

Different from Example 3, only primary crystallization was carried out. The scanning electron microscope image of the sample obtained after the primary crystallization in step (1) of Example 3 is shown in FIG. 4.

### Effect Test

### Test Example 1 Moisture Absorption Rate Test

The testing method is as follows: 10 new 20 mL glass vials were filled with the ergothioneine crystals obtained in Examples 1 to 4 and Comparative Examples 1 to 5 and a commercially available ergothioneine crystal, respectively. The glass vials were cleaned, dried in an oven at 105° C. to constant weights, and then numbered with their gram weights recorded. After the samples of examples and comparative examples and the commercially available product were dried to constant weights, the glass vials were each charged with 1 g of powder and placed in a constant temperature and humidity chamber (room temperature, 95% relative humidity). The samples were weighed at 24-hour intervals until the weights of the samples became constant. The moisture absorption rates were calculated. The results are shown in Table 2. The scanning electron microscope image of the commercially available ergothioneine crystal is shown in FIG. 5, and the powder X-ray diffraction pattern of the commercially available ergothioneine crystal is shown in FIG. 10. The following test examples all employed the above-mentioned commercially available product for testing.

TABLE 2
Moisture absorption rate test results

| | | | | | Compar- | Compar- | Compar- | Compar- | Compar- | |
|---|---|---|---|---|---|---|---|---|---|---|
| | | | | | ative | ative | ative | ative | ative | Commercially |
| | Exam- | Exam- | Exam- | Exam- | Exam- | Exam- | Exam- | Exam- | Exam- | available |
| | ple 1 | ple 2 | ple 3 | ple 4 | ple 1 | ple 2 | ple 3 | ple 4 | ple 5 | product |
| Moisture | 0.12 | 0.08 | 0.05 | 0.07 | 0.18 | 0.23 | 0.20 | 0.25 | 0.35 | 0.51 |
| absorption | | | | | | | | | | |
| rate (%) | | | | | | | | | | |

### Test Example 2 XRD Detection

Testing apparatus and its model: Rotating-anode X-ray diffractometer, D/Max-2500/PC.

Testing reference: 0451 X-ray diffraction method in Pharmacopoeia of the People's Republic of China, 2015 Edition, Volume IV.

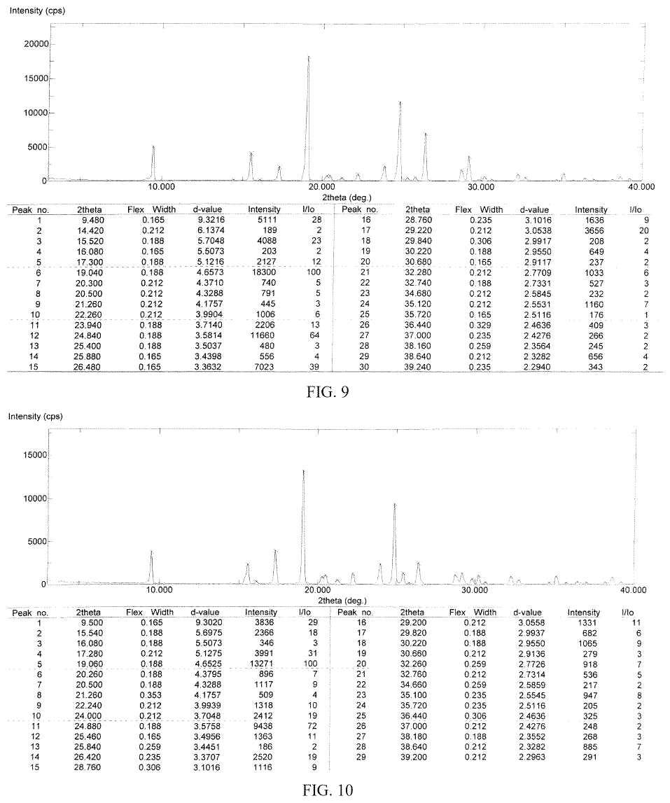

The detection results of Example 1 are shown in FIG. 6, the detection results of Examples 3 and 4 are shown in FIGS. 7 and 8, the detection results of Comparative Example 5 are shown in FIG. 9, and the detection results of the commercially available product are shown in FIG. 10.

### Test Example 3 Odor Detection and Photostability Detection

The testing method is as follows: All different ergothioneine samples were dried to constant weights, 20 mL transparent glass vials (with caps) were dried to constant weights, and the content of ergothioneine of each sample was measured, in triplicate per sample.

Each glass vial was charged with 1 g of sample powder to evaluate whether the samples emitted odor, and the evaluation results were recorded.

The glass vials were transferred to sunlight exposure conditions, and odor evaluation was conducted after 1 week and 2 weeks of sunlight exposure.

After 4 weeks of sunlight exposure, the content of ergothioneine in the powder was re-measured to determine the retention rate of ergothioneine.

Odor evaluation was conducted in the following method: Each sample was placed in a vial with a cap; after sunlight exposure, the vial was uncapped and transferred near the nose for odor evaluation, and each sample was evaluated by five individuals.

The retention rate of ergothioneine powder was determined in the following method.

1) Before sunlight exposure, the initial ergothioneine powder was dried to a constant weight, and 0.1 g of powder was dissolved in 100 mL of water. The ergothioneine concentration a1 was determined, and then the ergothioneine content was calculated according to the following formula:

$$Content = a 1 / 1 * 100 \% .$$

2) Content after 4 weeks of sunlight exposure: The ergothioneine powder exposed to sunlight for 4 weeks was dried to a constant weight, and 0.1 g of powder was dissolved in 100 mL of water. The ergothioneine concentration a2 was determined, and then the ergothioneine content was calculated according to the following formula:

$$3 ) Retention rate after 4 weeks of sunlight exposure = ( a 1 - a 2 ) / a 1 * 100 \% .$$

$$Content = a 2 / 1 * 100 \% .$$

The detection results are shown in Table 3.

TABLE 3
Odor detection and photostability detection results

| | | | | | Compar- | Compar- | Compar- | Compar- | Compar- | |
|---|---|---|---|---|---|---|---|---|---|---|
| | | | | | ative | ative | ative | ative | ative | Commercially |
| | Exam- | Exam- | Exam- | Exam- | Exam- | Exam- | Exam- | Exam- | Exam- | available |
| | ple 1 | ple 2 | ple 3 | ple 4 | ple 1 | ple 2 | ple 3 | ple 4 | ple 5 | product |
| Initial | No | No | No | No | No odor | No odor | No odor | No odor | No odor | No odor |
| smell | odor | odor | odor | odor | | | | | | |
| Smell | No | No | No | No | Very | Very | Very | Very | Very | Strong odor |
| after 1 | odor | odor | odor | odor | faint | faint | faint | faint | faint | resembling |
| week of | | | | | odor | odor | odor | odor | odor | trimethyl- |
| sunlight | | | | | | | | | | amine |
| exposure | | | | | | | | | | |
| Smell | Very | Very | Very | Very | Strong odor | Strong odor | Strong odor | Strong odor | Strong odor | Strong odor |
| after 2 | faint | faint | faint | faint | resembling | resembling | resembling | resembling | resembling | resembling |
| weeks of | odor | odor | odor | odor | trimethyl- | trimethyl- | trimethyl- | trimethyl- | trimethyl- | trimethyl- |
| sunlight | | | | | amine | amine | amine | amine | amine | amine |
| exposure | | | | | | | | | | |
| Retention | 96% | 95% | 99% | 97% | 86% | 85% | 89% | 90% | 90% | 75% |
| rate after | | | | | | | | | | |
| 4 weeks of | | | | | | | | | | |
| sunlight | | | | | | | | | | |
| exposure | | | | | | | | | | |
| (%) | | | | | | | | | | |

### Test Example 4 Thermogravimetric Analysis

Temperature range: room temperature to 800° C.; temperature control speed: 10° C./minute; atmosphere: nitrogen atmosphere.

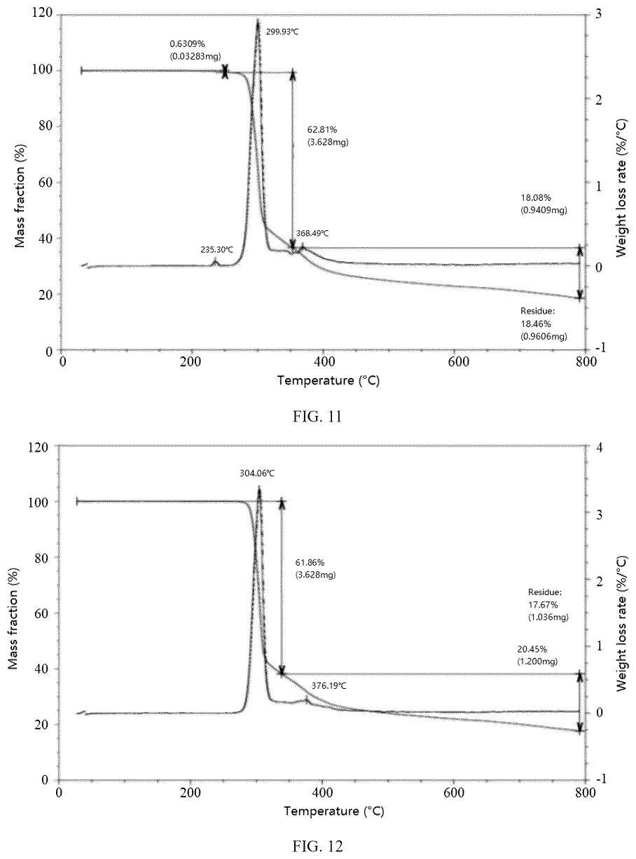

The detection results of Example 3 are shown in FIG. 11, and the maximum mass loss temperature was 304.06° C. The detection results of the commercially available product are shown in FIG. 12, and the maximum mass loss temperature was 299.93° C. The maximum weight loss temperatures of the crystal samples of the examples in the present disclosure are all higher than the maximum mass loss temperature of the commercially available product, indicating that the crystal samples in the present disclosure all have superior thermal stability.

### Test Example 5 Melting Point Test

The melting point determination for ergothioneine crystals was conducted according to GB/T 617-2006 4.2. The determined melting point/melting range of Example 3 was 275° C. to 277° C.

The preceding detailed description illustrates one feasible embodiment of the present disclosure. This embodiment is not intended to limit the patentable scope of the present disclosure. Any equivalent implementations or modifications made without departing from the spirit of the present disclosure shall be construed as falling within the scope of the technical solutions of the present disclosure.

What is claimed is:

1. A low-odor, high-stability ergothioneine crystal, wherein a powder X-ray diffraction pattern of the ergothioneine crystal, measured using Cu-Kα radiation and expressed in terms of 2θ, has characteristic peaks at least at 9.50±0.2°, 14.46±0.2°, 15.52±0.2°, 16.10±0.2°, 17.32±0.2°, 19.06±0.2°, 20.50±0.2°, 21.26-0.2°, 22.28-0.2°, 23.98±0.2°, 24.88-0.2°, 25.48±0.2°, 25.92±0.2°, 26.40±0.2°, 28.74±0.2°, 29.22±0.2°, 29.86-0.2°, 30.26-0.2°, 30.72±0.2°, 32.26-0.2°, 32.76-0.2°, 34.66-0.2°, 35.10-0.2°, 35.68-0.2°, 36.56-0.2°, 36.98-0.2°, 37.96±0.2°, 38.62-0.2°, and 39.24±0.2°.

2. The ergothioneine crystal according to claim 1, wherein a maximum mass loss temperature of the ergothioneine crystal is 304.06° C.

3. The ergothioneine crystal according to claim 1, wherein a melting point of the ergothioneine crystal is 275° C. to 277° C.

4. The ergothioneine crystal according to claim 1, wherein the ergothioneine crystal is prepared in the following method:
(1) dissolving crude ergothioneine in water to prepare a crude ergothioneine solution with a concentration of 110 g/L, adding methanol, controlling a temperature at 35° C., stirring at a rotational speed of 600 rpm to carry out primary crystallization, and maintaining crystal growth for 1 hour to obtain a crystal 1, wherein an added amount of methanol is 3.5 times a volume of the crude ergothioneine solution;
(2) re-dissolving the crystal 1 in water to obtain a solution A with a concentration of 400 g/L, and controlling the temperature at 60° C.; and
(3) adding methanol to the solution A, keeping the temperature unchanged, stirring at a rotational speed of 350 rpm until crystal precipitation occurs, maintaining crystal growth for 1.5 hours, continuously adding methanol, keeping the temperature unchanged, carrying out crystal growth for 2 hours, gradiently lowering the temperature at a rate of 10° C. per hour until the temperature is lowered to 35° C., collecting the crystal by filtration, and drying the crystal at 70° C. to obtain the ergothioneine crystal, wherein a total amount of methanol in two additions is 5 times a volume of the solution A.
